(19) United States
(12) Patent Application Publication
(10) Pub. No.: US 2024/0108567 A1
(43) Pub. Date: Apr. 4, 2024

(54) REDUCTION OF OIL TRANSFER FROM SCALP TO HAIR WITH INCREASING POLARITY OF HAIR

(71) Applicant: The Procter & Gamble Company, Cincinnati, OH (US)

(72) Inventors: Supriya Punyani, Singapore (SG); Husen Kartasasmita Kang, Singapore (SG)

(21) Appl. No.: 18/469,588

(22) Filed: Sep. 19, 2023

Related U.S. Application Data
(60) Provisional application No. 63/409,077, filed on Sep. 22, 2022.

(51) Int. Cl. A61K 8/72; A61Q 5/00; A61Q 5/12

(52) U.S. Cl. CPC A61K 8/72; A61Q 5/008; A61Q 5/12; A61K 2800/5426

(57) ABSTRACT

A hair care composition that includes 0.05% to 5% of a hydrophilic polymer selected from the group consisting of polyvinylalcohols, polyacrylates ethers, polyvinyl pyrrolidones, polyvinyl pyrrolidones copolymer, vinyl cyanides, vinyl phosphates, vinyl phosphonates, vinyl sulfates, vinyl sulfonates, polyamines, cellulose, collagen, polyethylene glycols, polycarboxylate co-polymers of maleic/olefin, and mixtures thereof. The hair care composition provides a water contact angle of <85 to hair reduces oil transfer to hair.

### FIELD

The present invention relates to a hair care composition for reducing oil transfer from scalp to hair.

### BACKGROUND

Scalp's oils affect many aspects of scalp and hair physiology as well as satisfaction with hair's look and feel. Perception of unwanted hair oiliness is impacted by several endogenous factors such as scalp type, hair type, hormones, age, diet, and exogenous factors such as hair care products, climate, and lifestyle. While today's shampoo products are designed to remove oil, people often feel dissatisfied with the length of time their hair feels clean. Shampoos do not typically affect the transfer of scalp oils from scalp to hair, which happens throughout the day and night. It has not been found that certain materials and formulations can modify the polarity of the hair surface, which helps reduce oil transfer from scalp to hair.

### SUMMARY

The present application is directed to a hair care products composition comprising a material or mixtures of materials which makes hair polar wherein one or more material is selected from synthetic or naturally derived hydrophilic polymers and/or cationic surfactants. The hair care composition can comprise about 0.05% to 5% of a hydrophilic polymer selected from the group consisting of polyvinylalcohols and derivatives, polyacrylates ethers, polyvinyl pyrrolidones, polyvinyl pyrrolidones copolymer (vinylpyrrolidone/dimethylaminoethylmethacrylate), vinyl cyanides, vinyl phosphates, vinyl phosphonates, vinyl sulfates, vinyl sulfonates, polyethylenimine and other polyamines, cellulose, collagen, polyethylene glycols and polycarboxylate co-polymers of maleic/olefin and mixtures thereof; wherein the hair care composition provides a water contact angle of les than 85 to hair. The total lipid value on hair decreases when compared to a control, the total lipid value on hair is less than 0.15 when measured using Fourier-Transform Infra-red spectroscopy (FTIR).

### DETAILED DESCRIPTION

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions herein can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the composition onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Rinse-off” in reference to compositions, means compositions intended to be applied to keratinous substrate and subsequently removed by washing, rinsing or wiping within a few minutes or less from the application. These “rinse-off” compositions are to be distinguished from “leave-on” compositions, which are intended to be applied to and allowed to remain longer on the keratinous tissue.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 5 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M. Wt.” as used herein refers to the weight average molecular weight unless otherwise stated. The weight average molecular weight may be measured by gel permeation chromatography. “QS” means sufficient quantity for 100%.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for use herein. Except if stated otherwise, the term “polymer” used herein includes any type of polymer including homopolymers and copolymers.

The mechanism of action for reducing oil transfer from scalp to hair involves surface modification of hair surface, creating polar surface (hydrophilic, high surface energy, lower water contact angle on hair). As hair growing out of scalp is hydrophobic in nature, after shower using a typical cleansing shampoo or hair products it retains same degree of hydrophobicity or increase in in hydrophobicity due to deposition of conditioning ingredients such as silicone, fatty alcohol. During the day and night, before the next wash the oil continuously transfer from scalp to hair and higher hydrophobic hair leads to more and faster oil transfer from scalp to hair. Currently technologies have been focusing on removing oil, reducing oil secretion from sebaceous glands which doesn't stop hair gets greasy during the day and night, as oil keep transferring from scalp to hair. Accordingly, it would be desirable to incorporate hydrophilic polymer(s) and/or cationic surfactant(s) into hair compositions to modify the polarity of the hair surface and help reduce oil transfer from the scalp to the hair. Reducing oil transfer improves hair feel by reducing or eliminating the greasy or oily feeling. This method of treatment makes hair cleaner longer achieved by making hair surface polar and reducing oil transfer from scalp to hair. Degree of hydrophilicity on hair can be determined by measuring water contact angle measurement with a mobile surface analyzer (MSA), which is a known technique in the art. The amount of oil reduction on hair can be measured using Fourier-Transform Infra-red spectroscopy (FTIR) as total lipids (CH₂), triglycerides, fatty acids or using a sebumeter, which is a known technique in the art.

### A. Hydrophilic Polymers

The hydrophilic polymer can be synthetic or naturally derived, can be one polymer or a mixture of two or more polymer or a copolymer with another polymeric unit. Hydrophilic polymers can include, but are not limited to, polyacrylates (and esters), other functionalized polyolefins, (such as PVA (polyvinyl alcohols and esters), PVA ethers, PVP (vinyl pyrrolidones), PVP copolymer (vinylpyrrolidone/dimethylaminoethylmethacrylate), vinyl cyanides, phosphates, phosphonates, sulfates, sulfonates, etc. polyethylenimine and other polyamines, polyethylene glycols and polycarboxylate co-polymers of maleic/olefin. The olefin segment may include a variety of linear, branched and cyclic alkenes. Suitable alkenes may include or be derived from propylene, ethylene, or butylenes. Particularly suitable alkenes may include or be derived from butylene, for example, isobutylene and diisobutylene. The hydrophilic polymer may have a cationic charge. The hydrophilic polymer may be polyquaterniu-10, such as UCARE Extreme Polymer supplied by Dow Inc., or may be cetyl hydroxyethylcellulose, such as Polysurf 67 CS or Natrosol 330 PLUS CS, both supplied by Ashland. Other suitable polymeric agents include silicone materials such as polydimethysiloxane materials (e.g., Wacker HC303 from Wacker Silicones), fluorinated acrylic copolymer e.g., Capstone ST100 and ST300 from Dupont), polycarboxylate copolymers (e.g., Acusol 460 from Dow), and acrylic polymers (Polyquart® Pro A from BASF, Rhoplex EZ Clean 200 from Dow, Polyquart® Pro, Polyquart® Ampho 149, and Polyquart® EcoClean from Cognis) and other polyethers, poly(styrene maleic anhydride), polyesters, polyureas, polyurethanes, polycarbonates, polyacrylamides, sugars and polymeric analogs, chitosan, collagen, tapioca starch and derivatives thereof. The hydrophilic polymer may be selected from PVP copolymer (Vinylpyrrolidone/Dimethylaminoethylmethacrylate), mono-long alkyl amidoamine salt or mixtures thereof. The hydrophilic polymer may be included in the composition at a level by weight of from about 0.05% to about 5%, or from about 0.05% to about 3%, or from about 0.1% to about 2%, and/or from about 0.2% to about 1%.

### B. Cationic Surfactant

The cationic surfactant can be one cationic surfactant or a mixture of two or more cationic surfactants. The cationic surfactant may be selected from mono-long alkyl quaternized ammonium salt, di-long alkyl quaternized ammonium salt, mono-long alkyl amidoamine salt or mixtures thereof. The cationic surfactant system can be included in the composition at a level by weight of from about 0.1% to about 10%, from about 0.5% to about 8%, from about 0.8% to about 5%, and from about 1.0% to about 4%.

The mono-long alkyl quaternized ammonium salt cationic surfactants useful herein are those having one long alkyl chain which has from about 12 to about 30 carbon atoms, or from about 16 to about 24 carbon atoms, and/or from about 18 to about 22 carbon atoms. The remaining groups attached to the nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. The counterion is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Nonlimiting examples of such mono-long alkyl quaternized ammonium salt cationic surfactants include: behenyl trimethyl ammonium salt.

The di-long alkyl quaternized ammonium salt cationic surfactants useful herein are those having two long alkyl chains of from 12 to 30 carbon atoms, or from 16 to 24 carbon atoms, or from 16 to 22 carbon atoms or an aromatic, alkoxy, polyoxyalkylene, alkylamido, hydroxyalkyl, aryl or alkylaryl group having up to about 30 carbon atoms. The remaining substituents on the nitrogen atom are selected from an aliphatic group of from 1 to about 8 carbon atoms, or from 1 to 3 carbon atoms or an aromatic, alkoxy, polyoxyalkylene, alkylamido, hydroxyalkyl, aryl or alkylaryl group having up to about 8 carbon atoms. The counterion is a salt forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Nonlimiting examples of di-long alkyl cationic surfactants include dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide

Materials may be chosen from hydrophilic polymers such as PVP, Polyquart Pro A, sodium hyaluronate and/or cationic surfactant such as stearamidopropyldimethylamine can also be used in the combination to increase the size of the benefit.

The concentration of the hydrophilic polymer in the present hair care product compositions may be from about 0.05% to 5%, or from about 0.05% to about 3%, or from about 0.1% to about 2%, and/or from about 0.2% to about 1%.

| | Total Lipid | Water |
|---|---|---|
| | measured | contact angle |
| | on Hair (CH₂ | on Hair (deg |
| Name (0.3% in ethanol:water) | using FTIR) | using MSA) |
| Oily hair (no treatment) | 0.114 | 92.6 |
| Ethanol:Water (1:1, vehicle only) | 0.113 | 85.7 |
| PVP | 0.082 | 54.9 |
| copolymer¹(vinylpyrrolidone/ | | |
| dimethylaminoethyl methacrylate) | | |
| Acrylic copolymer sodium salt² | 0.103 | 59.8 |
| Collagen³ | 0.097 | 82 |

¹Copolymer 845-O (PVP copolymer), Ashland
²Polyquart ^{®} Pro A, BASF
³Gelatin Novotec ^{®} CB800, Gelita

The Total Lipid on hair is measured using FTIR wherein it detects the vibration of CH₂ backbone as lipid group. CH₂ is the lipid functional group CH₂ carbon-hydrogen backbone.

The hair care composition may provide a water contact angle of <85 to hair, may provide a water contact angle of <80 to hair, or may provide a water contact angle of <75 to hair.

The hair care composition may provide a decrease in total lipid value on hair compared to a control. The hair care composition may provide a total lipid value on hair which is less than 0.15 (e.g., less than 0.11 or even less than 0.09) when measured using Fourier-Transform Infra-red spectroscopy (FTIR) as total lipids (CH₂).

The cationic surfactant system can be included in the composition at about 0.1% to about 10%, (e.g., 0.5% to 8%, 0.8% to 5%, or even 1.0% to 4%).

| | % Sebum | Water |
|---|---|---|
| | transferred | contact angle |
| Name (2% in aqueous | measured on hair | on Hair (degree |
| solution) | using sebumeter | using MSA) |
| Oily hair (no treatment) | 100% | 94 |
| Water (vehicle only) | 85% | 88 |
| Stearamidopropyl- | 56% | 71 |
| dimethylamine¹ | | |
| Benzyltriethylammonium | 65% | 78 |
| chloride² | | |
| Behenamidopropyl | 72% | 80 |
| dimethylamine³ | | |

¹Genamin SPA, Clariant KCI Ltd.
²SAPDMA, KCI, Ltd.
³KCI Ltd.

### FORMULATIONS AND EXAMPLES

The following are non-limiting examples. The examples are given solely for the purpose of illustration and are not to be construed as limitations, as many variations thereof are possible without departing from the spirit and scope of the invention, which would be recognized by one of ordinary skill in the art.

### Formulation Preparation and Treatment Protocols to Evaluate the Effectiveness of Hydrophilic Polymers and Cationic Surfactant

### Leave-On Treatment Composition Preparation:

The leave-on treatment compositions are prepared by adding the hydrophilic polymers and perfume, if needed, into ethanol/water carrier and stirred until complete dissolution. The solution pH is adjusted using sodium hydroxide (50% w/w) or citric acid to a final pH of 4.0-6.0. The Sepimax® Zen is then added, if needed, and the solution is mixed using a high-speed-mixer for 2-5 minutes at 1800-2300 rpm until a uniform composition is obtained.

### Leave-On Hair Treatment Protocol:

An amount of 0.20 g of each composition of Examples Ito IV is spread via a syringe onto separate natural virgin brown hair switches weighing 2.0 g (dosage 0.10 g of solution per g of hair). The hair is allowed to air dry and then oil is transferred from scalp mimic using the oil transfer method described above. The oil on hair surface is then measured using FTIR or sebumeter.

### Shampoo Composition Preparation:

The shampoo composition delivers consumer desired shampooing in addition to preventing oil transfer from scalp to hair.

The shampoo composition comprises from about 0.01% to about 5%, alternatively from about 0.05% to about 3%, alternatively from about 0.1% to about 2% of a compound selected from the group consisting of hydrophilic polymers and mixtures thereof, by weight of the shampoo composition. After applying to the hair a shampoo composition as described herein, the method then comprises rinsing the shampoo composition from the hair.

### Shampoo Hair Treatment Protocol:

All testing are performed on Virgin Caucasian Hair weighing approximately 2.0 grams and having a length of approximately 6 inches. The hair switches are commercially available from IHIP (International Hair Importers). Three hair switches 1 per rinse-off compositions per dosage are used. Each hair switch is washed with clarifying shampoo followed by a treatment with the rinse-off conditioner according to the following protocol. An amount of 0.20 g of shampoo is spread via a syringe onto separate hair switch. That is, the dosage is 0.10 g of shampoo per g of hair. Each application consists of adding shampoo to the hair, milking for 30 seconds followed by rinsing for 30 seconds. Excess water is squeezed from the hair switches.

### Rinse-Off Treatment Composition Preparation:

The rinse-off treatment compositions can be prepared by any conventional method well known in the art. The cationic surfactants and the fatty alcohols are mixed together and heated to from about 66° C. to about 85° C. to form an oil phase. Separately, the disodium EDTA, the methylchloroisothiazolinone (preservative) and the water are mixed and heated to from about 20° C. to about 48° C. to form an aqueous phase. The oil phase is mixed into the water phase under high shear to form the gel matrix. The remaining of the components are added into the gel matrix with agitation. Then, the composition is cooled down to room temperature.

### Rinse-Off Hair Treatment Protocol:

All testing are performed on Virgin Caucasian hair switches weighing approximately 2.0 grams and having a length of approximately 6 inches. The hair switches are commercially available from IHIP (International Hair Importers). Three hair switches per rinse-off compositions per dosage are used. Each hair switch is washed with clarifying shampoo followed by a treatment with the rinse-off conditioner according to the following protocol.

An amount of 0.20 g of clarifying shampoo is spread via a syringe onto separate hair switch. That is, the dosage is 0.10 g of shampoo per g of hair. Each application consists of adding shampoo to the hair, milking for 30 seconds followed by rinsing for 30 seconds. Excess water is squeezed from the hair switches and then 0.1 g/g of the rinse off hair treatment is applied and milked for 30 seconds and left on hair to rest for 15 minutes and then rinsed off with water for 30 seconds.

Oil Transfer from Scalp Mimic to Hair Switch:

Rectangular Scalp surface mimic (Bio Skin Plate #F-1, made of polyurethane elastomer material, length 192 mm, Width 129 mm, thickness 5 mm from Ohken Co., Ltd. Japan) is procured and washed with 15% SLS aqueous solution and air dried. 0.38 g of artificial sebum (from Advanced testing laboratories, Cincinnati) is applied using a finger glove and spread completely with light pressure to cover the full area of scalp mimic. The hair with treatment or without treatment is placed on scalp mimic and another scalp mimic is placed on top of hair switch, so that hair switch gets sandwiched between two scalp mimics. A gentle pressure for 5 seconds is applied and then hair is used to make measurement of oil transferred on hair from scalp mimic using FTIR or sebumeter (SEBUMETER® SM 815, Courage+Khazaka electronic GmbH). The % amount of oil transferred from scalp mimic to hair switch is calculated as follows:
A=Amount of oil measured using sebumeter on hair treated with composition containing the hydrophilic polymer and/or cationic surfactant
B=Amount of oil measured using sebumeter on hair treated with vehicle containing no hydrophilic polymer and/or cationic surfactant

% oil transfer from scalp mimic to hair=[(B−A)×100]/B

### Evaluation Methods

The hair switches that are treated with the shampoo, rinse-off conditioner, scrub, leave on treatment compositions are evaluated using the following methodology after and before oil transfer using scalp mimic.

a. Fourier Transform Infra-red (FTIR) Spectroscopy Measurement

Upon braiding, the hair switches are measured on 6 different locations in the middle of the switch with mid IR ATR-FTIR (Perkin Elmer Frontier FTIR with Specac Golden Gate single bounce diamond crystal ATR accessory). The measurement range is done from 4000-600 cm⁻¹ in absorption mode, with 8 scans and 4 cm⁻¹ spectral resolution. The ATR clamping pressure is controlled using pressure torque fixed at 70 Ncm for all the hair switches measurement. The spectrums are analyzed using MATLAB based code to preprocess the spectrum and calculate total lipid that is defined as ratio of CH₂ stretching peak area divided by Amide 2 peak area above the individual spectrum's baseline. The preprocessing includes the baseline correction and vector normalization. Vector normalization is calculated from the average absorption value of spectrum within selected spectral ranges. The average value calculated will then be subtracted from the spectrum, which center the spectrum around absorption value=0. This is followed by calculating the sum of squares of all absorption values, and respective spectrum is divided by the square root of this sum. The vector norm of the result spectrum is 1.

$$\begin{matrix}{a}_{m} = \frac{{\sum }_{k} a ( k )}{N} \\ {a}^{′} ( k ) = a ( k ) - {a}_{m} \\ a \text{"(} k \text{)=} \frac{{a}^{′} ( k )}{\sqrt{{\sum }_{k} {( {a}^{′} ( k ) )}^{2}}} \\ {\sum }_{k} ( a \text{"(} k ) {)}^{2} \text{=1}\end{matrix}$$

The CH₂ stretching peak area is obtained from the integration area under the absorption spectrum from right edge of 2946 cm⁻¹ to left edge of 2837 cm⁻¹ and above straight-line baseline, obtained from spectrum intersection at 3000 cm⁻¹ and 2700 cm⁻¹. The amide 2 peak area is obtained from the integration area under absorption spectrum from right edge of 1580 cm⁻¹ to left edge of 1475 cm⁻¹.
b. Sebumeter Measurement

Hair Switches (2 g, 10 inches, Caucasian virgin or damaged hair supplied by International Hair Importers) is placed on a clean surface and then sebumeter (Courage and Khazaka MPA 580 or 815 console) is used, where cartridge into the console establish the blank baseline. The cartridge with opaque film is put in contact with hair for 30 seconds firmly and tightly until 30 seconds counter goes to zero. Re-insert cartridge into the console and record the oil value displayed on screen. Move to new measuring section of the cartridge by depressing the slide of the cartridge and then pulling it back up, placing it at new area on hair 1 cm away from previous measure. Calculate the amount of oil transfer as shared above.

Hair Switch Look Assessment Method: The treated hair switches are kept at room temperature, lab conditions (32-35° C., 50% RH) for 2 hrs and then ten expert graders are asked to rate each of them in terms of hair oily look based on a 5 point scale, 5 being the highest (very oily) and 1 being the lowest rating (not oily).

### Formulation Examples to Evaluate the Effectiveness of Hydrophilic Polymers

### Leave-On Treatment Formulation:

| | Formula Example | | | | |
|---|---|---|---|---|---|
| | Leave-on | | | | |
| | treatment | | | | |
| | Control 1 | 2 | 3 | 4 | 5 |
| | (wt./ | (wt./ | (wt./ | (wt./ | (wt./ |
| Raw Material | wt.)% | wt.)% | wt.)% | wt.)% | wt.)% |
| Distilled Water | QS | QS | QS | QS | QS |
| Ethanol¹ | 50.00 | 50.00 | 50.00 | 50.00 | 50.00 |
| PVP | 0 | 0.3 | 0 | 0 | 0.15 |
| copolymer²(Vinyl- | | | | | |
| pyrrolidone/ | | | | | |
| Dimethylamino- | | | | | |
| ethylmethacrylate) | | | | | |
| Acrylic copolymer | 0 | 0 | 0.3 | 0 | 0.15 |
| sodium salt³ | | | | | |
| Collagen⁴ | 0 | 0 | 0 | 0.3 | 0 |
| % oil transferred | 65 | 21 | 45 | 37 | 15 |
| on hair versus | | | | | |
| Leave-on | | | | | |
| Treatment | | | | | |
| Control at dose | | | | | |
| of 0.10 g of | | | | | |
| composition | | | | | |
| for 1.0 g of hair | | | | | |
| (Lower is Better) | | | | | |
| using sebumeter | | | | | |
| Rating Hair Oily | 4 | 2 | 3 | 3 | 1 |
| look vs. Leave-on | | | | | |
| Treatment Control | | | | | |
| at dose | | | | | |
| of 0.10 g of | | | | | |
| composition | | | | | |
| for 1.0 g of hair. | | | | | |
| 5 for very oily and | | | | | |
| 1 for not oily | | | | | |

¹Alcodis (Brussels)
²Copolymer 845-O (PVP copolymer), Ashland
³Polyquart Pro A, BASF
⁴Gelatin Novotec CB800, Gelita

| Formula Example | 6 | 7 | 8 | 9 | 10 | 11 |
|---|---|---|---|---|---|---|
| Raw Material | | | | | | |
| Distilled Water | QS | QS | QS | QS | QS | QS |
| Ethanol | 25.0 | 25.0 | 50.0 | 50.0 | 50.0 | 50.0 |
| PVP copolymer¹(Vinylpyrrolidone/ | 0 | 0.25 | 0 | 0 | 0 | 0.25 |
| Dimethylaminoethylmethacrylate) | | | | | | |
| Acrylic copolymer sodium salt² | 0 | 0 | 0.25 | 0 | 0 | 0 |
| Stearamidopropyldimethylamine³ | 0 | 0 | 0 | 1 | 0 | 0 |
| Benzyltriethylammonium chloride⁴ | 0 | 0 | 0 | 0 | 1 | 0 |
| Decanediol (decylene glycol)⁵ | 1.0 | 1.0 | 1.0 | 1.0 | 1.0 | 1.0 |
| Silica⁶ | 3.0 | 3.0 | 3.0 | 3.0 | 3.0 | 3.0 |
| Tapioca Starch⁷ | 2.0 | 2.0 | 2.0 | 2.0 | 2.0 | 2.0 |
| Sepimax Zen⁸ | 0.25 | 0.25 | 0.25 | 0.25 | 0.25 | 0.25 |
| Salicylic acid⁹ | 0 | 0 | 0 | 0 | 0 | 0.5 |
| Propylene glycol¹⁰ | 0 | 0 | 0 | 0 | 0 | 0.5 |
| Piroctone Olamine¹¹ | 0 | 0 | 0 | 0 | 0 | 0.45 |
| Sucrose dilaurate¹² | 0.2 | 0.2 | 0.2 | 0.2 | 0.2 | 0.2 |
| Menthol¹³ | 0.3 | 0.3 | 0.3 | 0.3 | 0.3 | 0.3 |
| Citric acid¹⁴ | 0.05 | 0.05 | 0.05 | 0.05 | 0.05 | 0.05 |
| Perfume | 0.3 | 0.3 | 0.3 | 0.3 | 0.3 | 0.3 |
| Final pH | 4.5 | 4.5 | 4.5 | 4.5 | 4.5 | 4.2 |
| % oil transferred on hair versus | 47 | 20 | 27 | 35 | 40 | 15 |
| Leave-on Treatment Control at dose | | | | | | |
| of 0.10 g of composition for 1.0 g of | | | | | | |
| hair (Lower is Better) using | | | | | | |
| sebumeter | | | | | | |
| Rating Hair Oily look vs. Leave-on | 2.7 | 2 | 2 | 2.5 | 2.5 | 1 |
| Treatment Control at dose of 0.10 g | | | | | | |
| of composition for 1.0 g of hair. 5 | | | | | | |
| for very oily and 1 for not oily | | | | | | |

¹Copolymer 845-O (PVP copolymer), Ashland
²Polyquart Pro A, BASF
³Genamin, SPA, Clariant
⁴SAPDMA, KCI, Ltd.
⁵Symrise
⁶P&G Shiga Plant
⁷Dry Flo, Akzo Nobel
⁸Sepimax Zen, Seppic
⁹Novacyl
¹⁰Dow
¹¹Octopirox, Clariant
¹²BASF
¹³L-Menthol Flakes, BASF
¹⁴Jungbunzlauer Austria

Results: Formula 2 to 11 show reduction in % amount of oil transferred from scalp mimic to hair switch. Lower amount of oil are observed on hair treated with higher doses of leave-on Formulas 2 to 11. The hair oily look assessment results indicate addition of hydrophilic polymers and/or cationic surfactant results in less oily hair look versus no treatment and Example 6.

### Shampoo Composition

The method of reducing oil transfer described herein comprises applying to the hair a shampoo composition. The shampoo composition delivers consumer desired cleansing and potentially conditioning. It can also deliver reducing oil transfer in the case that it contains a hydrophilic polymer. The shampoo composition may comprise from about 0.01% to about 5% hydrophilic polymers selected from the group consisting of PVP copolymer, Jaguar excel, polyquart Pro A. After applying to the hair, a shampoo composition as described herein, the method then comprises rinsing the shampoo composition from the hair.

### Other Components in the Shampoo Composition

### A. Detersive Surfactant

The shampoo composition comprises one or more detersive surfactants, which provides cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the shampoo composition should be sufficient to provide the desired cleaning and lather performance, and generally ranges from about 2 wt % to about 50 wt %, from about 5 wt % to about 30 wt %, from about 8 wt % to about 25 wt %, from about 10 wt % to about 20 wt %, about 5 wt %, about 10 wt %, about 12 wt %, about 15 wt %, about 17 wt %, about 18 wt %, or about 20 wt %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the shampoo composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

The concentration of the surfactant in the composition should be sufficient to provide the desired cleaning and lather performance. The cleansing composition can comprise a total surfactant level of from about 6% to about 50%, from about 5% to about 35%, a total surfactant level of from about 10% to about 50%, by weight, from about 15% to about 45%, from about 15% to about 22%; from about 16% to about 20%; from about 17% to about 20%; by weight, from about 20% to about 40%, by weight, from about 22% to about 35%, and/or from about 25% to about 30%.

Suitable amphoteric or zwitterionic surfactants for use in the shampoo composition herein include those which are known for use in shampoo or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the shampoo composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present shampoo composition include cocoamphoacetate, cocoamphodiacetate, lauroamphoacetate, lauroamphodiacetate, and mixtures thereof.

Zwitterionic detersive surfactants suitable for use in the shampoo composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In some instances, zwitterionics such as betaines may be selected.

Non limiting examples of other anionic, zwitterionic, amphoteric or optional additional surfactants suitable for use in the shampoo composition are described in McCutcheon' s, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

The shampoo composition may also comprise a shampoo gel matrix, an aqueous carrier, and other additional ingredients described herein.

### Shampoo Formulation:

| Shampoo | Control | | | |
|---|---|---|---|---|
| Formula Example | A | B | C | D |
| Water Purified | Q.S to | Q.S to | Q.S to | Q.S to |
| | 100 | 100 | 100 | 100 |
| Sodium Lauryl Sulfate 29% | 20.0 | 20.0 | 20.0 | 13.0 |
| solution¹ | | | | |
| Cocamidopropyl betaine² | 3.5 | 3.5 | 3.5 | 0 |
| Sodium cocoyl isethionate³ | 0 | 0 | 0 | 2.5 |
| Sodium Lauroamphoacetate⁴ | 0 | 0 | 0 | 2.5 |
| Jaguar excel⁵ | 0.2 | 0.2 | 0.2 | 0.2 |
| Sodium hyaluronate⁶ | 0 | 0 | 0.3 | 0 |
| PVP copolymer⁷(Vinylpyrrolidone/ | 0 | 0.3 | 0 | 0 |
| Dimethylaminoethyl methacrylate) | | | | |
| Polyquaternium-10⁸ | 0 | 0 | 0 | 0.2 |
| Salicylic acid⁹ | 2 | 2 | 2 | 2 |
| Piroctone Olamine¹⁰ | 0.8 | 0.8 | 0.8 | 0.8 |
| Sodium Benzoate¹¹ | 0.25 | 0.25 | 0.25 | 0.25 |
| Sodium Hydroxide 50% Solution¹² | 0.19 | 0.19 | 0.19 | 0.19 |
| Menthol¹³ | 0.7 | 0.7 | 0.7 | 0.7 |
| Fragrance | 0.65 | 0.65 | 0.65 | 0.65 |
| % oil transferred on hair versus | 60 | 55 | 54 | 54 |
| Shampoo Control at dose of 0.10 g | | | | |
| of composition for 1.0 g of hair | | | | |
| (Lower is Better) using sebumeter | | | | |
| Rating Hair Oily look vs. Shampoo | 4 | 3 | 3 | 3 |
| Treatment Control at dose of 0.10 g | | | | |
| of composition for 1.0 g of hair. 5 | | | | |
| for very oily and 1 for not oily | | | | |

¹Zhishang Huizhou Petrochemicals
²Dehyton KE UP, BASF
³Jordapon Ci-Prill, BASF
⁴Miranol Ultra L-32, Solvay
⁵Solvay
⁶P&G Shiga Plant
⁷Copolymer 845-O (PVP copolymer), Ashland
⁸UCARE Extreme Polymer from Dow Inc.
⁹Novacyl
¹⁰Octopirox, Clariant
¹¹Emerald Kalama Chemicals
¹²Olin Chemicals, Rochester
¹³L-Menthol Flakes, BASF

Results: Above table represents shampoo compositions that are used for treating hair switches. As shown in the table, hair treated with these compositions has different amount of oil transferred from scalp mimic to hair switch. Hair treated with composition of B, C and D containing hydrophilic polymers makes has less oil transferred vs. control A and are rated low on oiliness look.

### Scrub Composition

Scrub composition and preparation is similar to shampoo preparation, with only addition of high salt such as sodium chloride as the last step in shampoo making.

### Examples of Scrub Formula

| | Control | | | |
|---|---|---|---|---|
| Material | E | F | G | H |
| Water | QS | QS | QS | QS |
| Sodium Lauryl Sulfate¹ | 13 | 13 | 13 | 12 |
| Sodium cocoyl isethionate² | 0 | 0 | 0 | 3.9 |
| Sodium Lauroamphoacetate³ | 0 | 0 | 0 | 2.2 |
| Cocamidopropyl Betaine⁴ | 2.3 | 2.3 | 2.3 | 0 |
| Glyceryl Stearate⁵ | 1 | 1 | 1 | 1 |
| Menthol⁶ | 0.35 | 0.35 | 0.35 | 0.35 |
| Perfume | 1.2 | 1.2 | 1.2 | 1.2 |
| Sodium hydroxide | 0.2 | 0.2 | 0.2 | 0.2 |
| (50% solution)⁷ | | | | |
| Sodium Chloride⁸ | 42.2 | 42.2 | 42.2 | 40 |
| Salicylic acid⁹ | 2 | 2 | 2 | 2 |
| Piroctone Olamine¹⁰ | 0.5 | 0.5 | 0.5 | 0.5 |
| Jaguar Excel¹¹ | | 0.5 | | |
| PVP copolymer¹² | | | 0.5 | |
| (Vinylpyrrolidone/ | | | | |
| Dimethylaminoethyl | | | | |
| methacrylate) | | | | |
| Polyquaternium-10¹³ | | | | 0.3 |
| Sodium Benzoate¹⁴ | 0.16 | 0.16 | 0.16 | 0.16 |
| % oil transferred | 60 | 54 | 53 | 54 |
| on hair versus Scrub | | | | |
| Control at dose of | | | | |
| 0.10 g of composition | | | | |
| for 1.0 g of hair | | | | |
| (Lower is Better) | | | | |
| using sebumeter | | | | |
| Rating Hair Oily look | 4 | 3 | 3 | 3 |
| vs. Scrub Control at | | | | |
| dose of 0.10 g of | | | | |
| composition for 1.0 g | | | | |
| of hair. 5 for very oily | | | | |
| and 1 for not oily | | | | |

¹Zhishang Huizhou Petrochemicals
²Jordapon Ci-Prill, BASF
³Miranol Ultra L-32, Solvay
⁴Dehyton KE UP, BASF
⁵Hallstar Italia
⁶L-Menthol Flakes, BASF
⁷Olin Chemicals, Rochester
⁸Merck KGan
⁹Novacyl
¹⁰Octopirox, Clariant
¹¹Solvay
¹²Copolymer 845-O (PVP copolymer), Ashland
¹³UCARE JR30M Extreme Polymer from Dow Inc.
¹⁴Emerald Kalama Chemical

Results: Above table represents scrub compositions that are used for treating hair switches. As shown in the table, hair treated with these compositions has different amount of oil transferred from scalp mimic to hair switch. Hair treated with composition of F, G and H containing hydrophilic polymers makes has less oil transferred vs. control E and are rated low on oiliness look.

### Conditioner Treatment Composition Having a Gel Matrix

The conditioner composition may comprise a gel matrix comprising (1) one or more high melting point fatty compounds, (2) a cationic surfactant system, and (3) an aqueous carrier. Cationic surfactant definition captured above.

### High Melting Point Fatty Compound

The high melting point fatty compound can be included in the composition at a level of from about 0.5%, or from about 1.0%, or form about 1.5%, or from about 2%, or from about 4%, and to about 15%, and/or from about 10% by weight of the composition. The high melting point fatty compound useful herein have a melting point of 25° C. or higher, or 40° C. or higher, or 45° C. or higher, or 50° C. or higher. The high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature.

Among a variety of high melting point fatty compounds, fatty alcohols are suitable for use in the conditioner composition. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

### Aqueous Carrier

The conditioner gel matrix of the leave-on treatment composition may include an aqueous carrier which may be water or a mixture of water and water-miscible solvents.

### Optional Ingredients in Rinse-Off Shampoo and Conditioner Formulations

### Rheology Modifier

The leave-on hair care composition may comprise a rheology modifier to increase the substantivity and stability of the composition. Any suitable rheology modifier can be used. The leave-on hair care composition may comprise 0.05% to 10% (e.g., 0.1%-10%, 0.5% to 2%, 0.7% to 2% or even 1% to 1.5%) of a rheology modifier. Some non-limiting examples of rheology modifiers are listed below.

The rheology modifier may be selected from homopolymers based on acrylic acid, methacrylic acid and the like (e.g., polyacrylate, polymethacrylate, polyethylacrylate, and polyacrylamide); alkali swellable and hydrophobically-modified alkali swellable acrylic copolymers or methacrylate copolymers (e.g., acrylic acid/acrylonitrogen copolymer, acrylates/steareth-20 itaconate copolymer, acrylates/ceteth-20 itaconate copolymer, acrylates/aminoacrylates copolymer, acrylates/steareth-20 methacrylate copolymer, acrylates/beheneth-25 methacrylate copolymer, acrylatesisteareth-20 methacrylate crosspolymer, acrylates/vinylneodecanoate crosspolymer, and acrylates/C10-C30 alkyl acrylate crosspolymer); crosslinked acrylic polymers (e.g., carbomers); alginic acid-based materials (e.g., sodium alginate and alginic acid propylene glycol esters); associative polymeric thickeners (e.g., hydrophobically modified cellulose derivatives, hydrophobically modified alkoxylated urethane polymers (e.g., PEG-150/decyl alcohol/SMDI copolymer, PEG-150/stearyl alcohol/SMDI copolymer and polyurethane-39); hydrophobically modified, alkali swellable emulsions (e.g., hydrophobically modified polyacrylates, hydrophobically modified polyacrylic acids, and hydrophobically modified polyacrylamides); hydrophobically modified polyethers (e.g., with a hydrophobe selected from cetyl, stearyl, or oleayl and a hydrophilic portion of repeating ethylene oxide groups (e.g., 10-300 repeating groups, 30-200 repeating groups or even 40-150 repeating groups (e.g., PEG-120-methylglucose dioleate, PEG-(40 or 60) sorbitan tetraoleate, PEG-150 pentaerythrityl tetrastearate, PEG-55 propylene glycol oleate, PEG-150 distearate)); cellulose and its derivatives (e.g., microcrystalline cellulose, carboxymethylcelluloses, hydroxyethylcellulose, hydroxypropylcellulose, hydroxypropylmethylcellulose, methylcellulose, ethylcellulose, nitro cellulose, cellulose sulfate, cellulose powder, and hydrophobically modified celluloses); guar and its derivatives (e.g., hydroxypropyl guar and guar hydroxypropyl trimonium chloride), polyalkyl oxides (e.g., polyethylene oxide, polypropylene oxide, POE-PPO copolymers); polyvinylpyrrolidone, crosslinked polyvinylpyrrolidone, polyvinyalcohol; polyethyleneimine; silicas (e.g., fumed silica, precipitated silica, and surface treated silica); water-swellable clays (e.g., laponite, bentolite, montmorilonite, smectite, and hectonite); gums (e.g., xanthan gum, guar gum, hydroxypropyl guar gum, Arabia gum, tragacanth, galactan, carob gum, karaya gum, and locust bean gum); dibenzylidene sorbitol; carrageenan; pectin; agar; quince seed (Cydonia oblonga Mill); starch (e.g., from rice, corn, potato and wheat); starch-derivatives (e.g. carboxymethyl starch, methylhydroxypropyl starch); algae extracts; dextran; succinoglucan; and pulleran,

Further non-limiting examples of rheology modifiers include acrylamide/ammonium acrylate copolymer (and)polyisobutene (and) polysorbate 20, acrylamide/sodium acryloyldimethyl taurate copolymer/isohexadecane/polysorbate 80, acrylates copolymer; acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil, C12-16 alkyl PEG-2 hydroxypropylhydroxyethyl ethylcellulose (HM-EHEC), carbomer, crosslinked polyvinylpyrrolidone (PVP), dibenzylidene sorbitol, hydroxyethyl ethylcellulose (EHEC), hydroxypropyl methylcellulose (HPMC), hydroxypropyl methylcellulose (HPMC), hydroxypropylcellulose (HPC), methylcellulose (MC), methylhydroxyethyl cellulose (MEHEC), PEG-150/decyl alcohol/SMDI copolymer, PEG-150/stearyl alcohol/SMDI copolymer, polyacrylamide/C13-14 isoparaffin/laureth-7; polyacrylate 13/polyisobutene/polysorbate 20; polyacrylate crosspolymer-6, polyamide-3; polyquaternium-37 (and) hydrogenated polydecene (and) trideceth-6, polyurethane-39, sodium acrylate/acryloyldimethyltaurate/dimethylacrylamide, crosspolymer (and) isohexadecane (and) polysorbate 60; sodium polyacrylate. Exemplary commercially-available rheology modifiers include ACULYN™ 28, Klucel M C S, Klucel H C S, Klucel G C S, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol Ulterez 30, Carbopol 1342, Sepigel™ 305, Simulgel™600, Sepimax Zen, and combinations thereof.

Some particularly suitable examples of rheology modifiers include derivatives of hydrogenated castor oil (e.g., hydroxystearin compounds) such as 12-hydroxystearic acid (cosmetic grade), available from Kawaken and CasChem, and trihydroxystearin available from Rheox or Elementis Specialies under the Thixcin® brand, Flowtone R available from ECC America, and Rheocin available from United Catalysts.

### Carrier

The compositions herein may be leave-on hair care compositions which may further include at least about 20 weight percent of an aqueous carrier. The aqueous carrier is prepared from dermatologically acceptable materials (e.g., demineralized or distilled water). The carrier may be water, organic solvents (miscible or non-miscible with water), silicone solvents or a mixture thereof. In some instances, the carrier may be water with minimal or no significant concentrations of organic solvent, except as otherwise incidentally incorporated into the composition as minor ingredients of other components. The carrier may include volatile solvents that have boiling points below or equal to 250° C.

Some non-limiting examples of carriers include water and solutions or mixtures of water with lower alkyl alcohols and/or polyhydric alcohols. Examples of lower alkyl alcohols are monohydric alcohols having 1 to 6 carbons such as ethanol, methanol, propanol, isopropanol, butanol, pentanol, and hexanol. Examples of polyhydric alcohols are glycols, such as dipropylene glycol, propylene glycol, butylene glycol, 1,4-butanediol, 3-allyloxy-1,2-propanediol, 1,2-hexanediol, 1,6-hexanediol, 1,3-butanediol, 1,3-propanediol, 2,2′-thiodiethanol, glycerin and other diols. Other non-limiting examples of organic solvents include polyglycols such as polypropylene glycol, polyethylene glycol, mixture of butylene glycol, polypropylene glycol, polyethylene glycol and ethers, such as dipropylene glycol n-butyl ether, sugars, and sugar derivatives.

### Silicones

The conditioning agent may be a silicone conditioning agent. The silicone conditioning agent may comprise volatile silicone, non-volatile silicone, or combinations thereof. The concentration of the silicone conditioning agent typically ranges from about 0.01% to about 10%, by weight of the composition, from about 0.1% to about 8%, from about 0.1% to about 5%, and/or from about 0.2% to about 3%. Non-limiting examples of suitable silicone conditioning agents, and optional suspending agents for the silicone, are described in U.S. Reissue Pat. Nos. 34,584, U.S. Pat. Nos. 5,104,646, and 5,106,609, which descriptions are incorporated herein by reference. The silicone conditioning agents can have a viscosity, as measured at 25° C., from about 20 to about 2,000,000 centistokes (“csk”), from about 1,000 to about 1,800,000 csk, from about 50,000 to about 1,500,000 csk, and/or from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

Silicone emulsions that may be suitable for use herein include, but are not limited to, emulsions of insoluble polysiloxanes prepared in accordance with the descriptions provided in U.S. Pat. No. 4,476,282 and U.S. Patent Application Publication No. 2007/0276087. Accordingly, suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The average molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscometer with spindle 6 at 2.5 rpm. The silicone emulsion may further include an additional emulsifier together with the anionic surfactant,

Other classes of silicones that may be suitable for use herein include but are not limited to: i) silicone fluids, including but not limited to, silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

### Organic Conditioning Materials

The conditioning agent may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### Anti-Dandruff Actives

The compositions herein may contain anti-dandruff agents. When present in these compositions, the anti-dandruff agent is typically included in an amount of about 0.01 wt. % to about 5 wt. %, based on the total weight of the hair care composition. In these compositions, the anti-dandruff particulate should be physically and chemically compatible with other ingredients of the composition, and should not otherwise unduly impair product stability, aesthetics, or performance.

Anti-dandruff agents suitable for use in hair care compositions include pyridinethione salts, hydroxy pyridones, such as piroctone olamine (octopirox), ciclopirox, rilopirox, and MEA-Hydroxyoctyloxypyridinone, azoles (e.g., ketoconazole, econazole, climbazole and elubiol), selenium sulfide, particulate sulfur, salicylic acid, and mixtures thereof. A typical anti-dandruff agent is as piroctone olamine (octopirox) or pyridinethione salt. Hair care compositions can also include a zinc-containing layered material. An example of a zinc-containing layered material can include zinc carbonate materials. Of these, zinc carbonate and pyridinethione salts (particularly zinc pyridinethione or “ZPT) are common in the composition, and often present together.

In addition to the anti-dandruff active, compositions may also include one or more anti-fungal or anti-microbial actives in addition to the metal pyrithione salt actives. Suitable anti-microbial actives include coal tar, sulfur, charcoal, whitfield's ointment, castellani's paint, aluminum chloride, gentian violet, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Typical anti-microbials include itraconazole, ketoconazole, selenium sulphide and coal tar.

### Hair Health Actives

A scalp health active may be included in the compositions herein to provide scalp benefits. This group of materials is varied and provides a wide range of benefits including anti-dandruff, anti-fungal, anti-microbial, moisturization, barrier improvement, and anti-oxidant, anti-itch, and sensates. Such skin health actives and scalp care actives include but are not limited to: zinc pyrithione, climbazole, octopirox, selenium sulfide, vitamin E and F, salicylic acid, glycols, glycolic acid, PCA, PEGs, erythritol, glycerin, lactates, hyaluronates, allantoin and other ureas, betaines, sorbitol, glutamates, xylitols, menthol, menthyl lactate, isocyclomone, benzyl alcohol, and natural extracts/oils including peppermint, spearmint, argan, jojoba and aloe.

### Optional Ingredients

The compositions herein suitable optional ingredients as desired. For example, the composition can optionally include other active or inactive ingredients.

The compositions may include other common hair ingredients such as other anti-dandruff actives, minoxidil, conditioning agents, and other suitable materials. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, rheology modifiers, hair conditioning agents, and surfactants.

The hair care compositions herein may be in the form of solutions, dispersion, emulsions, powders, talcs, encapsulated, spheres, spongers, solid dosage forms, foams, and other delivery mechanisms. The composition of the present invention may be rinse off shampoos and conditioners, hair tonics, leave-on hair products such as conditioners, treatment, and styling products, and any other form that may be applied to the hair.

TABLE 1
Rinse off Treatment Formulations

| | Control | Control | | | | |
|---|---|---|---|---|---|---|
| | I | J | Ex. K | Ex. L | Ex. M | Ex. N |
| | (wt/wt) | (wt/wt) | (wt/wt) | (wt/wt) | (wt/wt) | (wt/wt) |
| Formula | % | % | % | % | % | % |
| Stearamidopropyldimethyl | 1.95 | 0 | 1.95 | 1.95 | 0 | 0 |
| amine (SAPDMA)¹ | | | | | | |
| BTMS/IPA² | 0 | 1.95 | 0 | 0 | 1.95 | 1.95 |
| Cetyl Alcohol³ | 1.77 | 1.7 | 1.77 | 1.77 | 1.7 | 1.7 |
| Stearyl Alcohol⁴ | 3.05 | 4.3 | 3.05 | 3.05 | 4.3 | 4.3 |
| Disodium EDTA^{5y} | 0.13 | 0.1 | 0.13 | 0.13 | 0.1 | 0.1 |
| Benzyl Alcohol⁶ | 0.4 | 0.4 | 0.4 | 0.4 | 0.4 | 0.4 |
| Perfume | 1 | 0.7 | 1 | 1 | 0.7 | 0.7 |
| PVP | 0 | 0 | 0 | 0.3 | 0.3 | 0 |
| copolymer7(Vinylpyrrolido | | | | | | |
| ne/ | | | | | | |
| Dimethylaminoethyl | | | | | | |
| methacrylate) | | | | | | |
| Sodium Hyaluronate⁸ | 0 | 0 | 0.3 | 0 | 0 | 0.3 |
| DI Water | Q.S. | Q.S. | Q.S. | Q.S. | Q.S. | Q.S. |
| % oil transferred on hair | 66 | 72 | 57 | 56 | 68 | 67 |
| versus Rinse of treatment | | | | | | |
| respective control at dose | | | | | | |
| of 0.10 g of composition for | | | | | | |
| 1.0 g of hair (Lower is | | | | | | |
| Better) using sebumeter | | | | | | |
| Rating Hair Oily look vs. | 3 | 4 | 2 | 2 | 3 | 3 |
| Rinse of treatment | | | | | | |
| respective Control at dose | | | | | | |
| of 0.10 g of composition for | | | | | | |
| 1.0 g of hair. 5 for very oily | | | | | | |
| and 1 for not oily | | | | | | |

¹Genamin SPA, Clariant
²Supplied by Feixiang Chemicals (Zhangjingang) Co., Ltd.
³Supplied by P&G Chemicals
⁴Supplied by P&G Chemicals
⁵Trilon BD Powder supplied by BASF SE (Ludwigshafen, DE)
⁶Supplied by Ineos Maastricht BV (Maastricht NL)
⁷Copolymer 845-O (PVP copolymer), Ashland
⁸P&G Shiga Plant

Results: Table 1 represents rinse-off conditioner compositions that are used for treating hair switches. As shown in the table, hair treated with these compositions has different amount of oil transferred from scalp mimic to hair switch. Hair treated with composition of Control I makes has less oil transferred vs. control J, as SAPDMA makes hair more polar than BTMS. In addition Example K and L containing hydrophilic PVP copolymer and sodium hyaluronate in addition to SAPDMA further reduce % amount of oil transferred from scalp mimic to hair switch vs. control I without these hydrophilic polymers. Similarily, Example M and N containing hydrophilic PVP copolymer and sodium hyaluronate in addition to BTMS further reduce % amount of oil transferred from scalp mimic to hair switch vs. control J without these hydrophilic polymers.

### Regimen

Benefits for reducing oil transfer from scalp to hair may be achieved by a hair treatment regimen comprising a shampoo composition followed by treating the hair with rinse-off or leave-on composition or a scrub. In the present invention, the regimen may comprise both rinse-off and leave-on treatments with rinse-off and leave-on conditioner compositions.

TABLE 2
Regimen Examples at each step may contain 1 or more of hydrophilic polymers and cationic surfactant.

| | Regimen | Regimen | Regimen | Regimen | Regimen | |
|---|---|---|---|---|---|---|
| Regimen | Example | Example | Example | Example | Example | Regimen |
| Steps | 12 | 13 | 14 | 15 | 16 | Example 17 |
| Step 1 | Shampoo | Scrub* | Shampoo | Shampoo | Shampoo | Shampoo |
| | (Example | (Example H) | (Example | (Example | (Example | (Example D) |
| | D) | | D) | D) | D) | |
| Step 2 | Scrub* | Shampoo | Scrub* | Conditioner | Scrub* | Leave-on |
| | (Example | (Example D) | (Example | (Example | (Example | (Example |
| | H) | | H) | L) | H) | 11) |
| Step 3 | Conditioner | Conditioner | Leave-on | Leave-on | | |
| | (Example | (Example L) | (Example | (Example | | |
| | L) | | 11) | 11) | | |
| Step 4 | Leave-on | Leave-on | | | | |
| | (Example | (Example 11) | | | | |
| | 11) | | | | | |

*Scrub Treatment Time (T) and Dosage (D) may vary from regimen to regimen

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of Embodiments of the Invention are, in relevant part, incorporated herein by reference; the citation of any document is not to be construed as an admission that it is prior art. To the extent that any meaning or definition of a term in this document conflicts with any meaning or definition of the same term in a document incorporated by reference, the meaning or definition assigned to that term in this document shall govern.

While particular embodiments have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition, comprising:
about 0.05% to 5% of a hydrophilic polymer selected from the group consisting of polyvinylalcohols, polyacrylates ethers, polyvinyl pyrrolidones, polyvinyl pyrrolidone copolymers, vinyl cyanides, vinyl phosphates, vinyl phosphonates, vinyl sulfates, vinyl sulfonates, polyamines, cellulose, collagen, polyethylene glycols, polycarboxylate co-polymers of maleic/olefin and mixtures thereof, wherein the hair care composition provides a water contact angle of less than 85 to hair, when measured with a mobile surface analyzer.

2. A hair care composition according to claim 1, wherein there is a decrease in total lipid value on hair compared to a control using Fourier-Transform Infra-red spectroscopy method (FTIR).

3. A hair care composition according to claim 2 wherein the total lipid value on hair is less than 0.15 measured using FTIR.

4. A hair care composition according to claim 2 wherein the total lipid value on hair is less than 0.11 measured using FTIR.

5. A hair care composition according to Clam 2, wherein the total lipid value on hair is less than 0.09 measured using FTIR.

6. A hair care composition according to claim 1, wherein the hair care composition provides a water contact angle of less than 80 to hair.

7. A hair care composition according to claim 1, wherein the hair care composition provides a water contact angle of less than 75 to hair.

8. A hair care composition according to claim 1, wherein the hydrophilic polymer is about 0.05% to 3%.

9. A hair care composition according to claim 1, wherein the hydrophilic polymer is about 0.1% to 2%.

10. A hair care composition according to claim 1, wherein the hydrophilic polymer is about 0.2% to 1%.

11. A hair care composition according to claim 1, wherein the hydrophilic polymer is selected from the group consisting of vinylpyrrolidone/dimethylaminoethyl methacrylate acrylic copolymer, mono-long alkyl amidoamine salt, polyquaternium-10 and mixtures thereof.

12. A hair care composition according to claim 1, further comprising a surfactant.

13. A hair care composition according to claim 12, further comprising an anionic, amphoteric, nonionic or zwitterionic surfactant or mixtures thereof.

14. A hair care composition according to claim 1, further comprising a cationic polymer.

15. A hair care composition according to claim 1, further comprising about 0.01% to 5% of an anti-dandruff active selected from the group consisting of pyridinethione salts, hydroxy pyridones, azoles, selenium sulfide, particulate sulfur, salicylic acid, and mixtures thereof.

16. A hair care composition according to claim 15, wherein the hydroxyl pyridone is piroctone olamine.

17. A hair care composition according to claim 1, wherein the composition further comprises a conditioning agent.

18. A hair care composition according to claim 17, wherein the conditioning agent is a silicone.

19. A hair care composition according to claim 1, further comprising about 0.5% to about 7% of a perfume.
